(19) United States
(12) Patent Application Publication
(10) Pub. No.: US 2010/0198321 A1
(43) Pub. Date: Aug. 5, 2010

(54) THERMAL COUNTERPANE

(76) Inventor: Lidia Moeck, Hamburg (DE)

Correspondence Address: KELLY LOWRY & KELLEY, LLP, 6320 CANOGA AVENUE, SUITE 1650, WOODLAND HILLS, CA, 91367, US

(21) Appl. No.: 12/522,123

(22) PCT Filed: Jan. 4, 2008
(86) PCT No.: PCT/EP2008/000038

§ 371 Date: Apr. 7, 2010

(30) Foreign Application Priority Data
Jan. 5, 2007 (EP) 07000155.7

(51) Int. Cl. A61F 7/08

(52) U.S. Cl. 607/107

(57) ABSTRACT

The present invention relates to a heating blanket, particularly a heating sheet, having at least one inner layer which is constructed such that it is permeable for warm air and faces the body to be heated by means of the heating blanket, and at least one intermediate ply or intermediate layer for allowing warm air through, for conveying warm air through, for flowing warm air through and/or for transporting warm air. In order to replace an outer layer, which is composed of polyester and polyurethane, a coating is applied onto the intermediate layer.

### TECHNICAL FIELD

The present invention relates to a heating blanket, particularly a heating sheet, having
at least one inner layer which is constructed such that it is permeable for warm air and faces the body to be heated by means of the heating blanket; and
at least one intermediate ply or intermediate layer for allowing warm air through, for conveying warm air through, for flowing warm air through and/or for transporting warm air.

### PRIOR ART

Heating blankets of the type mentioned at the beginning primarily serve the temperature maintenance of a body positioned on the heating blanket and/or covered by the heating blanket in that warm air is supplied. Heating blankets of this type are used particularly, but not exclusively, in the medical field in order to prevent heat losses from people who are to be treated and whose independent thermoregulation is disturbed or is temporarily rendered ineffective.

The heating blanket is here laid under and/or over the body of the patient and the warm air which is supplied through at least one supply hose exits distributed over an area at the inner side of the heating blanket which faces the body, in order to heat the body.

The use of such heating blankets takes account of the fact that especially with regard to operations, that is to say in the medical and specifically in the surgical field, there exists an urgent requirement for continuous and “gentle” heat supply to the human body, as, due to the partial or full anaesthetisation which is generally required during an operation, fundamental bodily functions are rendered partially or completely ineffective for the duration of the anaesthetisation, so that the danger of dangerous cooling or hypothermia of the body is very high.

At least one intermediate layer is arranged between the at least one inner layer which faces the body and is constructed such that it is permeable for warm air and an outer layer which faces away from the body and is constructed such that it is substantially impermeable for warm air. This means that the interior space or “inner space” of the heating blanket, particularly of the heating sheet, is filled with a material which not only ensures an allowing through of warm air, but also guarantees a pressure-relieving positioning of anaesthetised patients and awake patients.

The heating sheet is thus laid under the body with its inner layer which faces the body uppermost and not only insulates the body, but heats it convectively, that is to say using warm air which is flowing out. Just by means of this convective heat supply, cooling or hypothermia even of a body to be operated on can be prevented in a reliable manner. A slight cooling is possible however.

The heating blankets known from the prior are therefore predominantly distinguished by the following structure:
as inner layer, at least one microfibre layer which is constructed such that it is air-permeable, that is to say is permeable for warm air;
as intermediate layer, at least one spacer/positioning woven or knitted fabric, particularly at least one woven fabric made from synthetic fibres or a woven or knitted polyester fabric, for allowing warm air through, conveying warm air through, flowing warm air through and or transporting warm air;
as outer layer, at least one textile layer which is constructed such that is air-impermeable, that is to say is impermeable for warm air. The outer layer is a separate layer which is predominantly composed of polyester and polyurethane coating.

It has been shown that the three-part configuration of the heating blanket, that is to say the production of an inner layer, an intermediate layer as well as an outer layer as separate layers which may be sewn together requires a complex production process and is in addition intensive in terms of material. On the other hand, it can still be concluded that, on account of the guaranteeing of a pressure relieving positioning of the patient, the intermediate layer fulfils a function which cannot be dispensed with. The fact that the inner layer consists of an air-permeable material means that the inner layer as well is of essential importance. An air- and moisture-impermeable outer layer, which is primarily ensured by the polyurethane integrated into the outer layer, is also additionally required for the functionality of the heating blanket.

### DESCRIPTION OF THE INVENTION, OBJECT, SOLUTION, ADVANTAGES

Starting from the presented disadvantages and discrepancies and also according to an evaluation of the prior art indicated, the object of the present invention in the case of heating blankets is to construct a heating blanket of the type mentioned at the beginning in such a manner that while protecting the functionality of the heating blanket, a separate layer can be dispensed with.

This object is achieved by a heating blanket with the features specified in claim 1. Advantageous configurations and expedient developments of the present invention are characterized in the dependent claims.

The basic idea of the invention is to apply a coating to the intermediate layer of a heating blanket directly. This coating can in particular be made of polyurethane. In this manner, a further separate layer is omitted for the heating blanket of the type mentioned at the beginning, which blanket is composed of polyester and polyurethane. At the same time however, the function of a separate outer layer sewn to the intermediate layer exists in that, for example with polyurethane, there is a material, which material is air- and water-impermeable. By applying the polyurethane to the intermediate layer, a coating is therefore created which replaces the separate outer layer of a heating blanket. The application of the polyurethane layer can in this case take place in the conventional manner in the spreading or reversal process. In this manner, the polyurethane enters into a direct combination with the intermediate layer, which layer can be composed of knitted fabric for example.

According to a preferred development of the present invention, the intermediate layer is at least one plastic woven or knitted fabric, which on the one hand is sufficiently elastic in order to guarantee a pleasant as well as pressure-relieving lying with respect to the deformation characteristics in the case of compressive stress, and which on the other hand is so rigid that it can only be compressed to a limited extent, so that the flowing through of warm air is guaranteed. The person skilled in the art in the field of heating blankets, particularly of heating sheets, for example a university of applied sciences engineer in the field of textile technology with special knowledge in the field of thermodynamics will furthermore know, with respect to the teaching of the present invention, to appreciate that, due to the arrangement of the intermediate ply or intermediate layer, the two other plies or layers (namely the inner ply or inner layer as well as the outer ply or outer layer, which is applied as a coating onto the intermediate layer) cannot be compressed to an extent which would no longer enable allowing warm air through, conveying warm air through, flowing warm air through and/or transporting warm air (the side body parts are likewise heated by the warm air flowing out of the inner ply or inner layer).

A practicable variant of the invention provides that a polyurethane foam is applied onto the intermediate layer. A further idea of the invention is to combine the polyurethane coating with the use of polyurethane foam.

In this case, the polyurethane can firstly be applied onto the intermediate layer in a certain layer thickness, wherein the polyurethane foam for forming a core is subsequently applied, in order then to once more apply a thin layer of polyurethane. What is produced is in one sense a sandwich structure. With the application of a first polyurethane layer, the intermediate layer is completely coated in order, as a result, to initially create an air-impermeable layer. With the polyurethane foam it is possible, in a short period of time, to build up larger layers. This cannot be achieved with a polyurethane coating, for example in the spreading or reversal process. The application of a further polyurethane layer serves then to form a smooth surface as well as the securing of the sealing of the sandwich structure. It is however also conceivable that the polyurethane foam is applied directly onto the intermediate layer. The fact that the polyurethane foam is applied directly onto the intermediate layer means that a faster buildup of a layer is ensured.

When producing the sandwich structure, it is possible to proceed in such a manner that the polyurethane layer be finished firstly, for example in the spreading or reversal process. Then, the polyurethane rigid foam is produced in a next step, for example by means of fluid raw components. Finally, the polyurethane rigid foam mixture is applied to the polyurethane layer and hardened. For forming the sandwich composite, a further polyurethane layer can, in addition, then be applied on the polyurethane foam in a further step. As the heating blanket should be constructed in a heat-reflecting manner on its layer which faces away from the body, it is advantageous that the polyurethane layer or the sandwich structure distinguishes itself by means of a white colouring.

In the context of the present invention, the heating blanket has
at least one head region, or at least one head part, and/or
at least one arm region, or at least one arm part, and/or
at least one chest region, or at least one chest part, and/or
at least one lower body region, or at least one lower body part, and/or
at least one leg region, or at least one leg part, and/or
at least one side part and/or
at least one pocket part.

These parts enable an optimal adaptation of the heating blanket to the body circumstances of the patient. So e.g. individual arms and/or individual legs can also be inserted into parts which are constructed in the manner of a pocket. It is also possible that entire body parts be covered.

An advantageous configuration of the invention provides that the heating blanket comprises at least one head region which can be brought into contact with the head and/or with the neck of a person covered by the heating blanket in order to heat the head and/or the neck by means of the warm air supplied.

The heating blanket according to the present invention does not therefore leave the head region and/or the neck region of a patient open, but rather includes these regions in the insulation and supplying of heat. In this manner, a considerable improvement in the temperature maintenance characteristics of the present heating blanket can be achieved.

A practicable variant of the invention provides that the heating blanket comprises at least one arm region which can be arranged in such a manner that it surrounds the arm or a part of the arm of a person covered by the heating blanket.

The arms of a patient can also be included in the thermoregulation completely or in part, for example merely in the upper arm region and/or merely in the lower arm region by means of the arm region or by means of the preferably two arm regions. This measure as well leads to a clear improvement of the thermic protection, as even by means of the (upper and/or lower) arm region a considerable heat loss can occur because the arms, like all extremities in general, are subject to the risk of a higher degree of cooling or of hypothermia compared to the trunk.

The heating blanket can also have at least one upper body region which can at least partially be brought into contact with the chest and/or at least partially be brought into contact with the back of a person to be positioned on the heating blanket and/or to be covered by the heating blanket for heating by means of escaping warm air and can particularly be arranged such that it at least surrounds the chest and/or at least partially surrounds the back.

The invention additionally provides that the heating blanket has at least one lower body region which can at least partially be brought into contact with the lower body and/or at least partially be brought into contact with at least one leg, particularly at least partially with one foot of a person to be positioned on the heating blanket and/or to be covered by the heating blanket for heating by means of escaping warm air and can particularly be arranged such that it partially surrounds the lower body and/or at least partially surrounds the leg or at least partially surrounds the foot.

A further advantageous configuration of the invention provides that the intermediate ply or intermediate layer and the coating are laminated with one another. Textile laminates, which are also designated as bonded fabrics, are multilayered flexible planar formed bodies which have at least one textile which is connected to at least one further layer over an area. A textile can likewise serve as the further layer. The laminate connection takes place by means of material engagement and can for example take place in such a manner that the layers, to which membranes also belong for example, are connected to one another in accordance with the melting or flame process. To this end, the surfaces of the layers to be laminated are melted with appropriate temperatures and connected under pressure. After cooling, a solid as well as flexible material is obtained, which material combines the characteristics of its starting materials. The laminate furthermore ensures the allowing of warm air through.

A practicable variant of the invention provides that the intermediate ply or intermediate layer and the coating are backed to one another. In contrast with lamination, backing represents the connecting of two or more woven fabric plies. The connection can take place by means of adhesive bonding. During the adhesive bonding, very thin adhesive films are used in accordance with the flame backing process (also designated as bonding). As foams are also meanwhile used for backing, backing and also the term lamination are also often used synonymously. In the case of backing, that is to say for example in the case of the adhesive bonding of two or a plurality of plies of the same or various textiles e.g. the following textile planar formed bodies can be used: woven fabrics, knitted fabrics, films, felts, paper, non-wovens, fleeces and foam films. If a foam or another non-textile planar formed body is connected to a textile surface, then one likewise talks of lamination. In a further field-specific terminology, one speaks of bonding when, in the context of thermal melting, the textile planar formed body is connected to a lining material by means of a binder (foam).

Preferably, the side of the knitted fabric which faces the inner layer is knitted very closely. A close knit is achieved in particular in that the stitch density and the fineness within the knitted fabric is set to be very fine.

In accordance with a further advantageous embodiment of the invention, the coating is present in the form of a woven fabric, particularly a polyester, and a wind/water membrane. The coating in the context of the invention can also be present in the form of a very close weave woven fabric, particularly in the form of a polyester.

The intermediate ply or intermediate layer is expediently provided with conductive threads. The advantage of providing the intermediate ply or intermediate layer with conductive threads can be seen in the fact that a source of warm air can be dispensed with. Rather, the heat is generated by the current flowing through the conductive fibres. The intermediate layer is therefore the heat source itself. An accumulator can be used as energy source. If the intermediate layer is present in the form of a knitted fabric, the conductive threads are worked in, inserted and positioned. The threads are preferably arranged in the intermediate layer towards the patient side. The conductive threads can also be woven into the intermediate layer. In the context of the invention the coating can also be non-slip.

### SHORT DESCRIPTION OF THE DRAWINGS

In the following, the invention is explained on the basis of the figures. In the figures:

FIG. 1 shows a schematic illustration of a plan view onto a heating blanket according to the invention

FIGS. 2 a) and b) show a schematic illustration of a sectional view of the heating blanket from FIG. 1 along the section line II-II from FIG. 1

### BEST WAY OF REALISING THE INVENTION

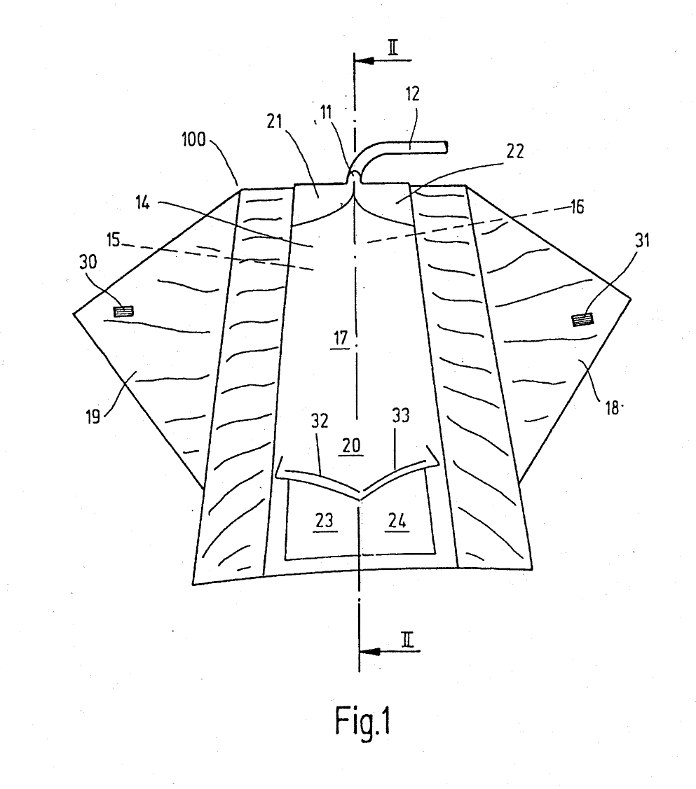

FIG. 1 shows an exemplary embodiment of a heating blanket 100 according to the invention intended for the body of a creature, particularly of a human, which heating blanket can in each case be used, for example in the case of operations on the body.

The heating blanket 100 has a connection 11 for an air hose 12 by means of which warm air from a warm air source, such as can be supplied for example from a current-driven heating apparatus or air-heating device.

The heating blanket 100 according to the invention has the feature that the warm air, after the switching on of the warm air source and after passing through the air hose 12 is distributed in the intermediate ply 16 which is constructed as an intermediate layer, which intermediate layer is introduced into the heating blanket 100, which overall is constructed as three ply, between the inner ply 14, which is constructed as an inner layer and faces the body, and the outer ply 15, which is constructed as an outer layer and faces away from the body, (the intermediate ply or intermediate layer 16 is therefore delimited in a manner essential to the invention by the inner ply or inner layer 14, which faces the body, as well as by the outer ply or outer layer 15, which faces away from the body). The inner layer or inner ply 14 is air- and moisture-permeable as well as hydrophilic. In order to guarantee this, the inner layer or inner ply 14 can consists of microfibre cloth and be closed, so that a fine distribution of the air is ensured. A strong air flow is therefore ruled out.

The provision of the intermediate ply 16 means that the warm air provided by the warm air source can in any event be guided continuously under the body to be warmed, so that the body parts laying on the heating blanket 100 can be heated reliably under any circumstances.

To this end, the internal space or “inner space” of the heating blanket 100 is filled with an intermediate-ply material made from a plastic woven fabric, namely from polyester knitted fabric; this material of the intermediate ply 16 is on the one hand so firm, stable and rigid that the allowing through of warm air is ensured in a manner essential to the invention, on the other hand even this material of the intermediate ply 16 has a degree of elasticity which guarantees a pressure-relieving positioning of anaesthetised patients, particularly of babies and of small children in a manner essential to the invention.

Furthermore, both the inner ply or inner layer 14 and the outer ply or outer layer 15 are not compressed by the arrangement of the intermediate ply or intermediate layer 16 to such an extent that allowing warm air through, conveying warm air through, flowing warm air through and/or transporting warm air is no longer possible (the side body parts are likewise warmed by means of the warm air flowing out of the inner ply 14).

The outer ply 15 of the heating blanket 100 is constructed in an airtight, moisture-tight and air-impermeable manner here, wherefore the outer ply 15 consists of a coating, preferably made from polyurethane, which coating is applied directly onto the intermediate layer 16. In principle, any woven fabric which is air- and moisture-impermeable, can be applied onto the intermediate layer, however. This also includes laminates or membrane coatings for example. In comparison with the heating blankets known from the prior art, the heating blanket 100 according to the invention therefore stands out in that the outer ply 15 in the form of a coating which is applied directly onto an intermediate layer replaces the outer ply made from a textile material of a heating blanket or heating sheet from the prior art. The inner ply 14 of the heating blanket 100 is air-permeable inasmuch as this inner layer 14 consists of microfibre material and, in a manner essential to the invention, has the most finely configured openings for flowing out the warm air supplied.

The previously mentioned technical measures lead to the fact that the warm air can essentially escape at the inner ply 14, which inner ply faces the body of a patient during the use of the heating blanket 100. In this manner, the heating blanket 100 functioning as a heating sheet can at the same time take care of an insulation against heat loss and additionally for active heat supply.

The air-permeable inner ply 14 of the heating blanket 100 is configured in the green shade which is conventional in operation fields. In contrast herewith, the airtight and air-impermeable outer ply 15 is in the form of a coating, preferably consisting of polyurethane, in a bright colour, preferably decorated in white, so that this outer ply 15 is less heat absorbing and also more heat reflecting and is consequently heated less strongly.

A cooler outer surface 15 additionally has the advantage that this outer surface 15 can less easily be dirtied by the residues of adhesive strips, tapes or the like during the fixing of the heating blanket 100, because the adhesive residues are less readily soluble at low temperatures and therefore substantially remain adhering to the adhesive strip, the tape or the like and simply not to the outer ply 14 of the heating blanket 100.

The reuseable heating blanket 100 is therefore intended for convective body heating and can, for example, be spread out under the body of the patient during operations.

The heating blanket 100 is shown in the spread out state. This heating blanket 100 has various, particularly shaped regions, wherein the “midpoint” of the heating blanket 100 forms an upper body region 17, which—as also in the case of known heating blankets—is to be arranged on the chest and/or under the back of a patient.

The upper body region 17 is continued by a lower body region 20; this lower body region 20 can cover the abdomen and also the hip part and also the posterior part and also the legs of a patient.

A two part head region 21, 22, which is constructed in a cowl-like manner, is also to be highlighted,
which head region can be brought partially into contact with the head and into contact with the neck of a person to be positioned on the heating blanket 100′ and to be covered by the heating blanket 100′ for heating by means of escaping warm air, and
which head region is constructed as a surrounding profile, partially for the head, and for the neck.

Furthermore, the heating blanket 100 has arm regions 18 and 19 constructed in the manner of side parts, which arm regions can be brought into contact with the right arm or with the left arm of a person to be positioned on the heating blanket 100 and to be covered by the heating blanket 100 for heating by means of escaping warm air in that the right arm is enclosed by the left arm region 18 in the plan view illustration of FIG. 1 and also the left arm is enclosed by the right arm region 19 in the plan view illustration of FIG. 1.

As the arm regions 18, 19 are essentially configured in the form of triangular tabs and consequently can also be folded or laid over a person who is lying on their back in relation to the plan view illustration according to FIG. 1, the arm regions 18, 19 also serve as a type of cover with which the heating blanket 100 is equipped.

Not least for this case of the use as a cover, the previously described arm regions 18, 19 have connection means in the form of hook-and-loop connections 30, 31 in their respective end regions (=region of the approximately right angle of the essentially triangular tabs), wherein two fleece-like hook-and-loop band sections 30 which are arranged at a distance from one another are assigned to the left arm region 18 in the plan view illustration of FIG. 1 and a hook-like hook-and-loop band section 31 corresponding hereto is assigned to the right arm region 19 in the plan view illustration of FIG. 1, by means of which hook-and-loop band sections the free ends of the arm regions 18, 19 can be fixed to one another to form a cover.

The two arm regions 18, 19 can be connected to one another by means of the connection means 30, 31 arranged at the ends after being laid around the body (=chest region and/or the back region) of the patient in order to form an upper body envelope of fixed length which keeps its configuration and position stable.

In this context, the hook-and-loop-like connection means 30, 31 at the ends of the two arm regions 18, 19 are arranged or set up in such a manner that the connection means 30, 31 allow a connection of the end regions of the arm regions 18, 19 at various angles; this makes it possible to position the two arm regions 18, 19 in various configurations and in various manners around the upper body or torso of the patient and yet to ensure a secure releasable connection of the end regions.

Finally, the heating blanket 100 has at least two mutually adjacent leg regions 23 and 24 which are of pocket-like construction and are located below the upper body region 17, that is to say in the lower body region 20, which leg regions are assigned to the right leg or the left leg of a person lying on their back in relation to the plan view illustration according to FIG. 1.

These leg regions 23 and 24 are constructed, in a manner essential to the invention, in the form of leg bags and/or can, in a manner essential to the invention, be releasably fixed to one another by means of suitable connection means, such as for example by means of hook-and-loop fasteners 32 and 33, as well as sealed, so that the warm air supplied remains in the region of the legs and is not lost to the outside.

The leg regions 23 and 24 can be brought at least partially in contact with the respective leg, particularly at least partially in contact with the respective foot, of a person to be positioned on the heating blanket 100 and to be covered by the heating sheet 100 for heating by means of escaping warm air, wherein the respective leg, particularly the respective foot is enclosed by the respective leg region 23 or 24.

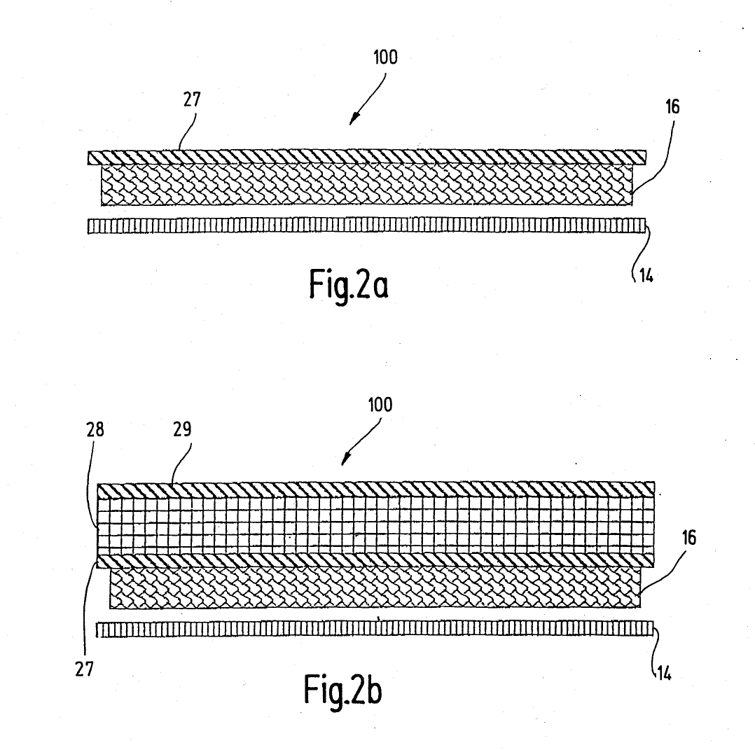

A section through a heating blanket 100 according to the invention, in the case of which the polyurethane is applied onto the intermediate layer 16, is shown in FIG. 2a). The intermediate layer 16 can be a woven fabric and/or a knitted fabric which can be sewn to the inner layer 14. The intermediate layer 16, which can be present in the form of a knitted fabric, is therefore delimited by the inner layer 14, which faces the body, and also by the polyurethane layer 27. A coating which replaces the separate outer layer 15 of the heating blanket 10 is therefore created by means of the application of the polyurethane onto the intermediate layer 16 and the thereby resulting polyurethane layer 27. The application of the polyurethane layer 27 can take place in the conventional manner in the spreading or reversal process. In this manner, the polyurethane enters into a direct combination with the intermediate layer 16, which layer is constructed of knitted fabric. It can be concluded that the polyurethane layer 27 can replace the entire outer layer 15 of the heating blanket 10 shown in FIG. 1. Thus, in the case of the conventional heating blanket 10, the outer layer 15 composed of polyurethane and polyester can henceforth be dispensed with. The polyurethane layer 27 can have a layer thickness of 1 to 5 mm. Polyurethane layers of this layer thickness stand out due to a high air-impermeability.

It can be drawn from FIG. 2b) that the intermediate layer 16, which can also be present here as a knitted fabric, can initially be completely coated with an application of a polyurethane layer 27, in order thereby to initially create an air-impermeable layer. The coating can also take place here in the spreading or reversal process. The polyurethane layer 27 can in this case be connected to a further layer which is composed of polyurethane foam 28. A polyurethane layer 27 is in turn applied onto the polyurethane foam 28. What is produced in this manner is in one sense a sandwich structure, which has a core with the polyurethane foam 28 and has top layers with the polyurethane layers 27. The polyurethane foam 27 which is applied directly onto the intermediate layer 16, which is constructed as a knitted fabric, initially constitutes the air-impermeable layer for the heating blanket 100. With the application of the polyurethane foam 28, it is then possible in a short space of time to build up larger layers which cannot be achieved by means of the spreading or reversal process. The application of a further polyurethane layer 27 serves the further sealing of the sandwich structure and thus also a further increased air-impermeability.

It is also possible in the context of the invention that the intermediate ply or intermediate layer 16 and the coating are laminated or backed to one another. Additionally, the side of the intermediate ply or intermediate layer 16 which is present as a knitted fabric and faces the inner layer can also be knitted very closely.

In order to be able to dispense with the warm air source 13 and the air hose 12 shown in FIG. 1, the intermediate ply or intermediate layer 16 of the heating blanket 100 can also be provided with conductive threads, the current source of which can in turn be an accumulator.

### REFERENCE LIST

100 Heating Blanket
10 Heating Blanket from the prior art
11 Connection
12 Air hose
13 Warm air source
14 Inner layer
15 Outer layer
16 Intermediate layer
17 Chest region
18 Arm region
19 Arm region
20 Lower body region
21 Head region
22 Head region
23 Leg region
24 Leg region
25 Hook-and-loop fastener
27 Polyurethane layer
28 Polyurethane foam
29 Polyurethane layer
30 Connection means
31 Connection means
32 Hook-and-loop fastener
33 Hook-and-loop fastener

1. A heating blanket, particularly a heating sheet, having
at least one inner layer which is constructed such that it is permeable for warm air and faces the body to be heated by means of the heating blanket; and
at least one intermediate ply or intermediate layer for allowing warm air through, for conveying warm air through, for flowing warm air through and/or for transporting warm air;
characterised in that a coating is applied onto the intermediate layer.

2. A heating blanket according to claim 1, characterised in that the coating is made from polyurethane.

3. A heating blanket according to claim 1, characterised in that the material of the intermediate layer is at least partially formed by at least one woven fabric and/or by at least one knitted fabric, particularly at least partially by at least one plastic woven fabric and/or by at least one plastic knitted fabric, specifically, at least partially by at least one polyester woven fabric and/or by at least one polyester knitted fabric.

4. A heating blanket according to claim 3, characterised in that a polyurethane layer is applied.

5. A heating blanket according to claim 3, characterised in that a polyurethane foam is applied.

6. A heating blanket according to claim 4, characterised in that polyurethane foam is applied onto the polyurethane layer and a second layer of polyurethane is applied onto the polyurethane foam.

7. A heating blanket according to claim 6, characterised in that the polyurethane layers have a layer thickness of 1 to 5 mm and the polyurethane foam is applied in a thickness of 1 to 3 cm.

8. A heating blanket according to claim 7, characterised in that the material of the intermediate layer
is both constructed so elastically that the body to be heated can be positioned in a pressure-relieving manner,
and is constructed so rigidly that the heating blanket can only be compressed by the body to be heated in a manner which allows allowing warm air through, conveying warm air through, flowing warm air through and/or transporting warm air through the intermediate layer.

9. A heating blanket according to claim 1, characterised in that the heating blanket has at least one head region,
which can at least partially be brought into contact with the head and/or at least partially be brought into contact with the neck of a person to be positioned on the heating blanket and/or to be covered by the heating blanket for heating by means of escaping warm air, and/or
which has at least one covering which is provided for suppressing the escape of warm air in certain regions and can be changed at positions, and/or
which is constructed as a surrounding profile, at least partially for the head, and at least partially for the neck of a person to be positioned on the heating blanket and/or to be covered by the heating blanket.

10. A heating blanket according to claim 1, characterised in that the heating blanket has at least one arm region which can at least partially be brought into contact with the arm or at least partially be brought into contact with both arms of a person to be positioned on the heating blanket and/or to be covered by the heating blanket for heating by means of escaping warm air and can particularly be arranged such that it partially surrounds the arm or at least partially surrounds both arms.

11. A heating blanket according to claim 1, characterised in that the heating blanket has at least one upper body region which can at least partially be brought into contact with the chest and/or at least partially be brought into contact with the back of a person to be positioned on the heating blanket and/or to be covered by the heating blanket for heating by means of escaping warm air and can particularly be arranged such that it at least surrounds the chest and/or at least partially surrounds the back.

12. A heating blanket according to claim 1, characterised in that the heating blanket has at least one lower body region which can at least partially be brought into contact with the lower body and/or at least partially be brought into contact with at least one leg, particularly at least partially with one foot of a person to be positioned on the heating blanket and/or to be covered by the heating blanket for heating by means of escaping warm air and can particularly be arranged such that it at least partially surrounds the lower body and/or at least partially surrounds the leg or at least partially surrounds the foot.

13. A heating blanket according to claim 3, characterised in that the intermediate ply or intermediate layer and the coating are laminated to one another.

14. A heating blanket according to claim 3, characterised in that the intermediate ply or intermediate layer and the coating are backed to one another.

15. A heating blanket according to claim 14, characterised in that the side of the knitted fabric which faces the inner layer is knitted very closely.

16. A heating blanket according to claim 13, characterised in that the coating is present in the form of a woven fabric, particularly a polyester, and a wind/water membrane.

17. A heating blanket according to claim 13, characterised in that the coating is present in the form of a very close weave woven fabric, particularly in the form of a polyester.

18. A heating blanket according to claim 1, characterised in that the intermediate ply or intermediate layer is provided with conductive threads.

19. A heating blanket according to claim 1, characterised in that the coating is non-slip.
